(19) United States
(12) Patent Application Publication
(10) Pub. No.: US 2008/0131383 A1
(43) Pub. Date: Jun. 5, 2008

(54) Cosmetic Composition For Obtaining A Cosmetic Coating With A Metallically Glossy Appearance, Artificial Fingernails And Use Of PVD Aluminum Pigments

(76) Inventors: Peter Kruger, Schnaittach (DE); Thomas Voit, Auerbach (DE)

Correspondence Address: OSTROLENK FABER GERB & SOFFEN, 1180 AVENUE OF THE AMERICAS, NEW YORK, NY, 100368403, US

(21) Appl. No.: 11/792,207

(22) PCT Filed: Jul. 26, 2006
(86) PCT No.: PCT/EP06/07354

§ 371 Date: May 31, 2007

(30) Foreign Application Priority Data
Jul. 29, 2005 (DE) 10 2005 036 333.4

(51) Int. Cl. A61K 8/26; A61Q 3/02

(52) U.S. Cl. 424/61; 514/769

(57) ABSTRACT

The invention relates to a cosmetic composition comprising a liquid phase and a PVD aluminum pigment, wherein the cosmetic composition contains a PVD aluminum pigment at a pigmentation level of from 0.05 to 5.0% by weight, based on the total weight of the cosmetic composition, and at least one leafing additive. The invention further relates to an artificial fingernail and to the use of a PVD aluminum pigment.

The invention relates to a cosmetic composition for producing a cosmetic coating having a lustrous metallic appearance.

A nail polish that imparts a metallic appearance to a fingernail is disclosed in WO 02/03913. PVD pigments are used in this known nail polish formulation. PVD pigments have a pronounced mirror-like luster because of their extremely smooth surface, although they have a non-leafing effect when applied to substrates. This means that they sink down in the liquid enamel film, thereby reducing the brilliant effect.

US 2004/0241423 also describes a nail polish showing a mirror effect.

As indicated in paragraph [0004] of the description, an improved mirror effect is achieved over WO 02/03913 particularly due to the fact that the preparation has a low viscosity and a high content of metal particles.

However, considering the high price of PVD pigments, it would be desirable to achieve satisfactory results using a low content of metal pigments. Moreover, improved orientation, but no leafing effect, is achieved by the procedure described in US 2004/0241423.

The need still exists for cosmetic formulations that produce an extreme mirror-like luster after a coating has been applied.

It is an object of the present invention to provide a cosmetic composition that produces a stronger mirror-like luster after application than is possible with the cosmetic compositions known hitherto, giving the impression of a preferably closed metal film.

The object of the invention is achieved by providing a cosmetic composition comprising a liquid phase and PVD aluminum pigment, which cosmetic composition contains the PVD aluminum pigment at a pigmentation level of from 0.05 to 5.0% by weight, based on the total weight of the cosmetic composition, and at least one leafing additive.

Preferred developments are defined in the subclaims.

After the cosmetic composition of the invention has been applied, a cosmetic coating having a lustrous metallic appearance can be created.

Starting with known PVD-aluminum pigments (for example Metalure®, marketed by ECKART GmbH & Co. KG) which display a mirror-like gloss despite their non-leafing behavior, leafing characteristics are imparted to the pigments by adding one or more leafing additives, which causes the pigments to float on the surface of the enamel film to display a greatly improved high mirror gloss. The leafing additive binds to the surface of the PVD aluminum pigment, whereupon the PVD aluminum pigments are oriented on the surface or near the surface of a liquid.

The object is further achieved by the provision of an artificial fingernail as claimed in claim 21. For the purposes of the invention, the term “artificial fingernail” may also be interpreted as meaning an artificial toenail.

The object of the invention is also achieved by the use of PVD aluminum pigment showing leafing properties in a cosmetic composition, preferably a nail polish.

A preferred variant is defined in subclaim 22.

The term PVD aluminum pigment as applied to the invention means that the aluminum pigment was produced using a PVD process (PVD=Physical Vapor Deposition). Very thin aluminum pigments having an extremely smooth and reflective surface can be produced by PVD processes. PVD processes for the production of aluminum pigments are well known to the person skilled in the art, e.g., from EP 0 826 745, which is included herein by reference.

The PVD aluminum pigments used in the present invention have no diffracting structure or diffracting elements. The surface of the PVD aluminum pigments used in the present invention are smooth and flat and therefore produce the desired metallic mirror-like gloss.

These PVD aluminum pigments are preferably handled in highly dilute dispersions in organic solvents to avoid strong agglomeration.

It has now been found, very surprisingly, that leafing properties can be imparted by adding leafing additives to these PVD aluminum pigments, which always display non-leafing properties.

These pigments include a commercially available PVD aluminum pigment such as, for example, Metalure®, marketed by ECKART GmbH & Co. KG, and it was not to have been expected by the person skilled in the art that a leafing effect would be developed in a cosmetic composition by the simple addition of a leafing-promoting substance or a leafing additive.

Such leafing-promoting substances (“leafing additives”) are preferably long-chained phosphoric acid esters or mixtures of phosphoric acid esters. According to the invention phosphoric acid esters having a carbon chain length of from 12 to 24 carbon atoms have proven to be highly suitable. Especially preferred are phosphoric acid esters or mixtures of phosphoric acid esters in which the carbon chain has a length of from 14 to 18 carbon atoms. The carbon chain may be linear or branched. Preferably, the carbon chain is linear.

The phosphoric acid esters may include mono-, di-, and/or tri-esters, phosphoric acid monoesters being especially preferred.

The phosphoric acid esters are preferably fatty acid phosphoric acid esters, preferably fatty acid phosphoric acid monoesters.

Myristic, pentadecanoic, palmitic, margaric and stearic acid phosphoric acid esters have been found to be highly suitable and may be used individually or intermixed as the leafing additive.

It has been found, very surprisingly, that the desired effect (leafing effect) can be achieved merely by simple addition of the leafing additive to the dispersion of the PVD pigment. Neither is it necessary, surprisingly, to coat the PVD aluminum pigments with the leafing additive prior to the addition thereof to the cosmetic composition.

The invention further relates to a cosmetic composition which, after application, forms a coating preferably having the appearance of a closed metal film having a mirror-like luster never achieved hitherto.

For the purposes of the invention, a “cosmetic composition” refers to all cosmetic formulations having a liquid phase in which the PVD aluminum pigments display leafing properties and which are applied to a substantially flat substrate. The cosmetic preparations are preferably selected from the group consisting of liquid makeup, liquid eyeliner, liquid lid shadow, body lotion, perfume, lip gloss and nail polish.

The cosmetic composition is preferably a nail polish.

The lustrous appearance of the cosmetic composition of the invention appears following application of the cosmetic composition to a flat substrate. The effect is not necessarily visible in the same way in the cosmetic composition itself. Following application of the cosmetic composition of the invention, the PVD aluminum pigments float up to the surface of the preferably liquid enamel film on, say, natural or artificial fingernails, on the lips, eyelids, cheeks, or other flat areas of the body. In order to permit this orientation of the aluminum pigments it is necessary for the cosmetic composition to have a liquid phase.

Cosmetic compositions that contain no liquid phase are largely unsuitable, since a liquid phase is necessary to enable the aluminum pigments to float up and cause the desired metallic lustrous effect in the applied coating.

This means that, to cause orientation of the aluminum pigments, a content of moisture or solvent is needed in the cosmetic composition that is sufficient to permit an orientation of the effect pigments following application of the composition and evaporation of the solvent. The orientation of the PVD aluminum pigment can be further improved or assisted if the cosmetic composition is subjected to a mechanical influence while it is being applied. This happens, for example, when a nail polish is applied with a brush to a natural or artificial fingernail. For example, orientation is assisted during application of a lip gloss to the lips by the action of the applicator ball or roller ball of the lip gloss applicator stick. As an alternative, the lip gloss can alternatively be applied to the lips with a brush.

The liquid phase used is preferably a liquid component that does not jeopardize health when used in the prescribed manner. That means that, in the case of nail polish, organic solvents may be used which, in the case of a lip gloss, may be used either not at all or only to a restricted extent. The person skilled in the art, however, will have no difficulty in selecting a liquid phase which is suitable for the intended use, e.g., an organic solvent, water, oil, gel, or suitable mixtures thereof.

A nail polish composition according to the invention usually consists of components that are non-toxic for the intended purpose and are suitable for forming a film coating showing a metallic luster effect on natural or artificial fingernails. The term “non-toxic” here pertains in general to the substances listed in the EC Guideline 76/768/EC.

A nail polish composition preferably contains a solvent, at least one film-forming component, a PVD aluminum pigment, and a leafing additive.

The cosmetic composition of the invention for production of a cosmetic coating having a highly lustrous metallic appearance comprises a liquid phase and PVD aluminum pigment, which PVD aluminum pigment displays leafing properties and is present in the cosmetic composition at a pigmentation level of from 0.05 to 5.0% by weight, based on the total weight of the cosmetic composition, and also a leafing-promoting additive.

The pigmentation level of the PVD aluminum pigment is from 1 to 2% and preferably from 1.3 to 1.8% by weight, based on the total weight of the cosmetic composition.

The solids content of the cosmetic composition is preferably between 5 and 17% by weight, based on the total weight of the cosmetic composition.

The cosmetic composition of the invention is preferably a nail polish. It may contain a film-forming component selected, for example, from the group consisting of nitrocellulose, polyester resins, polyvinyl resins, alkyd resins, epoxy resins, or cellulose acetate butyrate and suitable non-toxic derivatives, and mixtures thereof. Cellulose acetate butyrate and nitrocellose are preferred. The nitrocellulose preferably has a molecular weight of at least 56,000 g/mol and more preferably at least 112,000 g/mol. Cellulose acetate butyrate preferably has a molecular weight of more than 83,000 g/mol.

For the production of a cosmetic composition, the PVD aluminum pigment is preferably used in the form of a dispersion in an organic solvent to which a leafing additive is added.

These are preferably long-chained (C12-C24) phosphoric acid esters, C14-C18 phosphoric acid esters being especially preferred. The desired effect (leafing effect) can be achieved by the simple addition of the leafing additive to the dispersion of the PVD aluminum pigment. This dispersion is then preferably allowed to stand for some time before further processing.

The solids content of the dispersion to be added to a cosmetic composition may be from 1 to 15% and preferably from 3 to 10% by weight, based on the total weight of the cosmetic composition. At higher concentrations of PVD aluminum pigment there is a risk of agglomeration of the aluminum pigments.

Regarding the selection of the solvent, it has been found, surprisingly, that ketones and acetates having a molecular weight below 120 g/mol are particularly suitable for achieving a leafing effect. For example, methyl isobutyl ketone, methyl ethyl ketone, ethyl acetate, butyl acetate, methyl acetate, acetone, or mixtures thereof may be used. Methyl ethyl ketone and ethyl acetate are preferred for nail polish compositions.

In some cases it may be necessary to match the solvent to the leafing additive employed.

Cosmetic compositions prepared according to the invention, especially nail polish compositions, display a hitherto unknown luster as a result.

But other solvents, such as toluene, ethanol, hexane, heptane, cyclopentane, cyclohexane, cyclic ethers, such as tetrahydrofuran or 1,4-dioxane, cellosolve acetate, ethyl cellosolve, butyl cellosolve, and mixtures thereof may also be used as organic solvents in the nail polish composition.

The solvent content of the nail polish compositions of the invention is preferably from 50 to 90%, more preferably from 60 to 85%, and even more preferably from 65 to 75% by weight, always based on the total weight of the nail polish composition.

Thus it is possible to obtain nail polish compositions that display a hitherto unknown mirror-like gloss when applied.

According to a preferred development of the invention, the nail polish composition may contain a plasticizer and/or a dispersing agent.

Plasticizers soften the binder, in particular, and make it easier to fabricate. Use may be made of a single plasticizer or a combination of more than one plasticizer. Examples of such plasticizers are camphor, castor oil, esters of citric, stearic, oleic, phthalic, and benzoic acids, and derivatives thereof.

The phthalate used, is preferably dibutyl phthalate, diethyl phthalate, diamyl phthalate, dioctyl phthalate, dibutoxyethyl phthalate, or a mixture thereof. As an alternative to the toxicologically unacceptable phthalates, however, use may be made of other plasticizers, such as, for example, glyceryl triacetate, glyceryl tripropionate, glyceryl tribenzoate, dibutyl tartrate, benzyl benzoate, tricresyl phosphate, tributyl phosphate, triphenyl phosphate, butyl acetylricinoleate, butyl stearate, triethyl citrate, acetyl tributyl citrate, saccharose-acetate isobutyrate, butyl glycolate, glycerin triacetate, and glycerin tripropionate, or a mixture thereof.

According to another embodiment of the invention, dispersing additives may be added in order to improve the dispersion of the PVD aluminum pigments. Preferred examples of such dispersing agents are montmorillonite clays such as bentonite and especially stearylalkonium hectorite and/or stearylalkonium bentonite. Another dispersing agent that can be used is polymeric urea, optionally in combination with bentonites.

Other additives that may be included in the cosmetic composition are other effect pigments and/or dyes such as colored pigments and/or dyestuffs.

The nail polish composition according to the invention may also contain other additives such as thixotropic agents, antioxidants, emulsifiers, vitamins, scents, antihalation [light shielding] agents, preservatives, fillers, and/or medicaments. These additives are known in the prior art and are optionally added in the usual quantities.

The nail polish composition of the invention, preferably consisting of non-toxic components, contains PVD-aluminum pigment having a metallic aluminum content of from 90 to 99.9% by weight, based on the total weight of the aluminum pigment, at a pigmentation level of from 0.05 to not more than 5% by weight, based on the total weight of the nail polish, and at least one leafing additive. A pigmentation level of from 0.1 to 2% by weight, based on the total weight of the nail polish, is even more preferred.

It has been found that the PVD aluminum pigment in the cosmetic composition can be used with very high degree of efficiency, ie, they display outstanding covering power. Cosmetic compositions containing a PVD aluminum pigment have an extremely high covering power. For this reason, the PVD aluminum pigment may be used to advantage at very low pigmentation levels in the cosmetic composition of the invention.

Aluminum pigment produced by PVD processes, because of their low thickness, are so flexible that they can adapt themselves perfectly to the shape of the substrate, ie, cling, so to speak, to the substrate.

It has been found with the present invention that the tendency of agglomeration to take place in the cosmetic composition, preferably a nail polish or a lip gloss, is surprisingly low when at least one leafing additive is incorporated therein.

In addition, the composition according to the invention may also contain additives such as perfumes, antioxidants, antihalation agents [light screening agents] or preservatives. Common substances known to the art are used here.

### EXAMPLES

### Nail Polish Composition

### Example 1 According to Invention

| | | Concentration |
|---|---|---|
| No. | Substance | in % w/w |
| 1 | Metalure ® CA-41010 AE | 19 |
| 2 | Hostaphat CS 120 * | 1 |
| 3 | Methyl ethyl ketone | 20 |
| 4 | Methyl isobutyl ketone | 20 |
| 5 | CAB 381.2 | 8 |
| 6 | Butyl acetate 98/100 | 32 |

* Stearylphosphoric acid ester

### Manufacture:

To an initial batch of butyl acetate the CAB powder is added portionwise with stirring. Components 2 to 4 are added successively to the Metalure® dispersion and gently blended.

### Comparative Example 2

| | | Concentration |
|---|---|---|
| No. | Substance | in % w/w |
| 1 | Metalure ® CA-41010 AE | 19 |
| 2 | Methyl ethyl ketone | 21 |
| 3 | Methyl isobutyl ketone | 20 |
| 4 | CAB 381.2 | 8 |
| 5 | Butyl acetate 98/100 | 32 |

Manufacture: As Example 1 but without the addition of Hostaphat CS 120

### Comparison and Assessment of the Compositions of Examples 1 and 2

Visual appearance of Examples 1 and 2 following application to an artificial fingernail:
Example 1 according to invention. Highly lustrous, silvery, closed metal film resembling a “liquid metal.”
Comparative Example 2: Shiny silver, clearly less luster than in Example 1.

1. A cosmetic composition comprising a liquid phase and a PVD aluminum pigment, characterized in that the cosmetic composition contains a PVD aluminum pigment at a pigmentation level of from 0.05 to 5.0% by weight, based on the total weight of the cosmetic composition, and at least one leafing additive.

2. The cosmetic composition as defined in claim 1, characterized in that said leafing additive is at least one long-chain phosphate ester.

3. The cosmetic composition as defined in claim 24, characterized in that said leafing additive is at least one long-chain phosphate ester having a carbon chain comprising from 14 to 18 carbon atoms.

4. The cosmetic composition as defined in claim 1, characterized in that that the leafing additive is a fatty acid phosphoric acid ester or a mixture of different fatty acid phosphoric acid esters.

5. The cosmetic composition as defined in claim 1, characterized in that that the pigmentation level of the PVD aluminum pigment is from 1 to 2% by weight and preferably from 1.3 to 1.8% by weight, based on the total weight of the cosmetic composition.

6. The cosmetic composition as defined in claim 1, characterized in that that the solids content of the cosmetic composition is between 5 and 17% by weight, based on the total weight of the cosmetic composition.

7. The cosmetic composition as defined in claim 1, characterized in that the cosmetic composition additionally contains at least one film-forming component and is a nail varnish.

8. The cosmetic composition as defined in claim 7, characterized in that said film-forming component comprises cellulose nitrate.

9. The cosmetic composition as defined in claim 8, characterized in that said film-forming component comprises cellulose nitrate having a molecular weight of more than 56,000 g/mol.

10. The cosmetic composition as defined in claim 7, characterized in that said film-forming component comprises cellulose acetate butyrate.

11. The cosmetic composition as defined in claim 1, characterized in that said liquid phase is an organic solvent.

12. The cosmetic composition as defined claim 11, characterized in that said organic solvent is selected from the group consisting of methylisobutyl ketone, methyl ethyl ketone, ethyl acetate, butyl acetate, methyl acetate, acetone, and mixtures thereof.

13. The cosmetic composition as defined in claim 12, characterized in that said organic solvent is methyl ethyl ketone or ethyl acetate.

14. The cosmetic composition as defined in claim 1, characterized in that said cosmetic composition additionally contains a plasticizer and/or a dispersing agent.

15. The cosmetic composition as defined in claim 14, characterized in that said cosmetic composition contains as plasticizer at least dibutyl phthalate.

16. The cosmetic composition as defined in claim 14, characterized in that said cosmetic composition contains, at least one dispersing agent selected from the group consisting of a bentonite, stearylalkonium hectorite or stearylalkonium bentonite, and polymeric.

17. The cosmetic composition as defined in claim 1, characterized in that said cosmetic composition additionally contains at least one coloring agent.

18. The cosmetic composition as defined in claim 17, characterized in that said coloring agent is a colored pigment and/or a dye.

19. The cosmetic composition as defined in claim 1, characterized in that said cosmetic composition contains additives, selected from the group consisting of thixotropic agents, antioxidants, emulsifiers, vitamins, perfumes, light-stabilizing agents, preservatives, fillers, and medicaments.

20. The cosmetic composition as defined claim 1, characterized in that the pigmentation level of the PVD aluminum pigments is from 0.2 to 2.0% by weight based on said total weight of said composition.

21. A synthetic fingernail, characterized in that said synthetic fingernail is coated with the cosmetic composition as defined in claim 1.

22. A method of use of a cosmetic composition comprising PVD aluminum pigment having leafing properties, comprising the steps of: preparing the cosmetic composition as defined in claim 1, and coating a nail with said composition as a nail varnish.

23. The method as defined in claim 22, characterized in that the PVD aluminum pigment is provided with a leafing additive on its surface.

24. The cosmetic composition as defined in claim 2, wherein said at least one long-chain phosphate ester has a carbon chain comprising from 12 to 24 carbon atoms.

25. The cosmetic composition as defined in claim 9, wherein said cellulose nitrate has a molecular weight of more than 112,000 g/mol.

26. The cosmetic composition as defined in claim 10, wherein said cellulose acetate butyrate has a molecular weight of more than 83,000 g/mol.

27. The cosmetic composition as defined in claim 11, wherein said organic solvent is selected from the group consisting of ketones, acetates having a molecular weight below 120 g/mol, and mixtures thereof.

28. The cosmetic composition as defined in claim 15, wherein said cosmetic composition further contains saccharose acetate isobutyrate as plasticizer.

29. The cosmetic composition as defined in claim 20, wherein said pigmentation level is from 0.3 to 1.0% by weight, based on said total weight of said composition.
